(19) United States
(12) Patent Application Publication
(10) Pub. No.: US 2016/0139039 A1
(43) Pub. Date: May 19, 2016

(54) IMAGING SYSTEM AND IMAGING METHOD

(71) Applicants: NATIONAL INSTITUTE OF ADVANCED INDUSTRIAL SCIENCE AND TECHNOLOGY, Tokyo (JP); NIKON CORPORATION, Tokyo (JP)

(72) Inventors: Yuzuru IKEHARA, Tsukuba-shi (JP); Mutsuo OGURA, Tsukuba-shi (JP); Susumu MAKINOUCHI, Tokyo (JP)

(73) Assignees: NATIONAL INSTITUTE OF ADVANCED INDUSTRIAL SCIENCE AND TECHNOLOGY, Tokyo (JP); NIKON CORPORATION, Tokyo (JP)

(21) Appl. No.: 14/951,934

(22) Filed: Nov. 25, 2015

Related U.S. Application Data
(63) Continuation of application No. PCT/JP2014/064282, filed on May 29, 2014.

(30) Foreign Application Priority Data
May 30, 2013 (JP) 2013-113825

(51) Int. Cl. G01N 21/359; A61B 5/00; H04N 5/33; H04N 5/232; G01N 21/3563; H04N 13/02

(52) U.S. Cl. CPC G01N 21/359; G01N 21/3563; H04N 13/02; H04N 5/33; H04N 5/23229; A61B 5/0077

(57) ABSTRACT

An imaging system includes an infrared camera 10 that is sensitive to light beams having wavelengths in an infrared region, a lighting unit 20 that emits light beams having multiple wavelengths in an infrared region in a region including the wavelengths to which the infrared camera is sensitive, and a control unit 30 that controls capture of an image by the infrared camera 10 and emission of a light beam by the lighting unit 20.

### CROSS REFERENCE TO RELATED APPLICATION

This is a Continuation of PCT Application No. PCT/JP2014/064282, filed on Dec. 4, 2014. The contents of the above-mentioned application are incorporated herein by reference.

### BACKGROUND

1. Field of the Invention

The present invention relates to an imaging system and an imaging method.

2. Description of Related Art

There are known imaging systems that capture images of a part of an animal or human body, or the like and use the images for various types of diagnosis or examination, observation, or other purposes. Such an imaging system applies light beams having predetermined wavelengths to the target area and captures images of the light beams reflected therefrom or transmitted therethrough. Desirably, such an imaging system can easily capture images of the inside of a living body. When a light beam having a wavelength of 1 μm or less is used, a high-resolution silicon image sensor can be used. Accordingly, there are being developed examination support devices or operation support devices that include such an image sensor and use near-infrared light beams having wavelengths of 1 μm or less. Such devices include those which use a photoabsorption band or fluorescence near 700 to 900 nm attributable to heme contained in a living body, an administered indocyanine dye, or the like. The usage of those devices is being explored in order to detect or evaluate anatomical information required for diagnosis or treatment or detect or evaluate a pathological condition and the spread thereof. Further, there is being realized a device for visualizing blood vessels, which are difficult to observe by oxygen metabolism monitoring or in a direct view when light is absorbed (see Non-Patent Literature 1).

On the other hand, the photoabsorption bands of main molecules included in a living body, such as water, lipid, and glucose, lie in near- and mid-infrared wavelength regions having wavelengths of 1 μm or more and 2.5 μm or less. For example, the photoabsorption band of water has peaks near wavelengths of 1500 nm and 2000 nm. A less absorptive wavelength region of about 700 to 1400 nm is called a “biological window.” The water content of each organ of a body slightly varies with the type of cells forming the organ or the pathological condition of the organ. An MRI T2 (proton)-weighted image, which uses such differences in water content, is used in examination or diagnosis. As with an MRI T2 (proton)-weighted image, near- and mid-infrared wavelength regions of 1 μm or more and 2.5 μm or less, in which the photoabsorption efficiency significantly varies, can serve as means with which the state of each organ can be evaluated.

These wavelength regions can also indicate the contents of lipid, glucose, and the like, which have different absorption peaks. Accordingly, it is expected that information indicating an image of a pathological tissue, such as irritation, cancer, degeneration, or regeneration, will be obtained from these wavelength regions. With regard to organ identification using near- and mid-infrared wavelength regions, there has been reported an example in which a hyperspectral camera including a spectral grating is used (see Non-Patent Literature 2).

There are also examples in which sharp images of blood vessels in a deep part of a living body are captured using a filter wheel provided with a lamp and a bandpass filter (see Patent Literatures 1, 2).

### CITATION LIST

### Patent Literature

[Patent Literature 1] Japanese Patent No. 5080014

[Patent Literature 2] Japanese Unexamined Patent Application Publication No. 2004-237051

### Non-Patent Literature

[Non-Patent Literature 1] Goro Nishimura, Journal of Japanese College of Angiology, Japanese College of Angiology, 2009, vol. 49, 139-145.

[Non-Patent Literature 2] Hamed Akbari, Kuniaki Uto, Yukio Kosugi, Kazuyuki Kojima, and Naofumi Tanaka, “Cancer detection using infrared hyperspectral Imaging,” Cancer Science, 2011, vol. 102, no. 4, 852-857.

However, any device or method requires a mechanical drive apparatus to obtain spectral information and therefore it takes time to capture an image. Further, the light source continuously emits light and therefore thermal effect is unavoidably exerted on the area to be observed. Further, the light source cannot be turned on and off quickly and therefore it is difficult to remove noise from the image sensor and thus to obtain a high SN ratio. Further, a hyperspectral camera having a spectral function has problems, including its expensiveness and the conflict between the wavelength resolution and camera sensitivity.

Further, it takes time to capture images of light beams having multiple wavelengths, thereby losing simultaneity. This becomes an obstacle when a stereo camera obtains a stereoscopic view based on a parallax.

### SUMMARY

A first aspect of the present invention provides an imaging system including an infrared camera that is sensitive to light beams having wavelengths in an infrared region, a lighting unit that emits light beams having multiple wavelengths in an infrared region in a region including the wavelengths to which the infrared camera is sensitive, and a control unit that controls capture of an image by the infrared camera and emission of a light beam by the lighting unit.

A second aspect of the present invention provides an imaging method including emitting light beams having multiple wavelengths in an infrared region toward a subject and capturing images of the subject using the light beams having the wavelengths.

A third aspect of the present invention provides an imaging system for capturing an image of a living body tissue. The imaging system includes a lighting unit that emits infrared light beams having wavelengths in an infrared region based on spectral properties of water and lipid, an infrared camera that receives the infrared light beams, a visible lighting unit that emits a visible light beam having a wavelength in a visible region, a visible camera that receives the visible light beam, and a control unit including an image processing unit that processes infrared images captured by the infrared camera using a visible image captured by the visible camera.

### BRIEF DESCRIPTION OF DRAWINGS

FIG. 1 is a diagram showing an example of an imaging system according to a first embodiment.

FIG. 2 is a perspective view showing an example of a lighting unit.

FIG. 3 is a diagram showing an example of a drive circuit of the lighting unit.

FIG. 4 is a function block diagram showing the imaging system shown in FIG. 1.

FIG. 5 is a diagram showing an operation sequence of the imaging system shown in FIG. 1.

FIG. 6 is a drawing showing an example of an image captured by the imaging system.

FIG. 6A is a graph showing absorption properties of water and lipid in a near-infrared region.

FIG. 6B(a) is a photograph captured by a visible light camera of pancreas, spleen, mesentery, and lymph node removed from a mouse, and FIG. 6B(b) is a photograph of these tissues taken by an InGaAs infrared camera which is sensitive to wavelengths of up 1600 nm, using a light beam having a wavelength of 1600 nm emitted from an LED.

FIG. 6C includes photographs taken by an InGaAs infrared camera which is sensitive to up 1600 nm and showing a mouse subjected to laparotomy, in which FIG. 6C(a) is a photograph taken using a light beam having a wavelength of 1050 nm emitted from an LED, and FIG. 6C(b) is a photograph taken using a light beam having a wavelength of 1600 nm emitted from an LED.

FIG. 7 is a diagram showing an example of an imaging system according to a second embodiment.

FIG. 8 is a diagram showing an example of an imaging system according to a third embodiment.

FIG. 9 is a diagram showing an example of an imaging system according to a fourth embodiment.

FIG. 10 is a diagram showing an example of an imaging system according to a fifth embodiment.

FIG. 11 is a diagram showing an example of an imaging system according to a sixth embodiment.

FIG. 12 is a diagram showing an example of an imaging system according to a seventh embodiment.

FIG. 13 is a diagram showing an example of an imaging system according to an eighth embodiment.

FIG. 14 is a diagram showing an example of an imaging system according to a ninth embodiment.

FIG. 15 is a diagram showing an example of an imaging system according to a tenth embodiment.

### DETAILED DESCRIPTION OF EMBODIMENTS

Now, embodiments of the present invention will be described with reference to the drawings. However, the present invention is not limited to the embodiments. To clarify the embodiments, the drawings are scaled as necessary, for example, partially enlarged or highlighted.

### First Embodiment

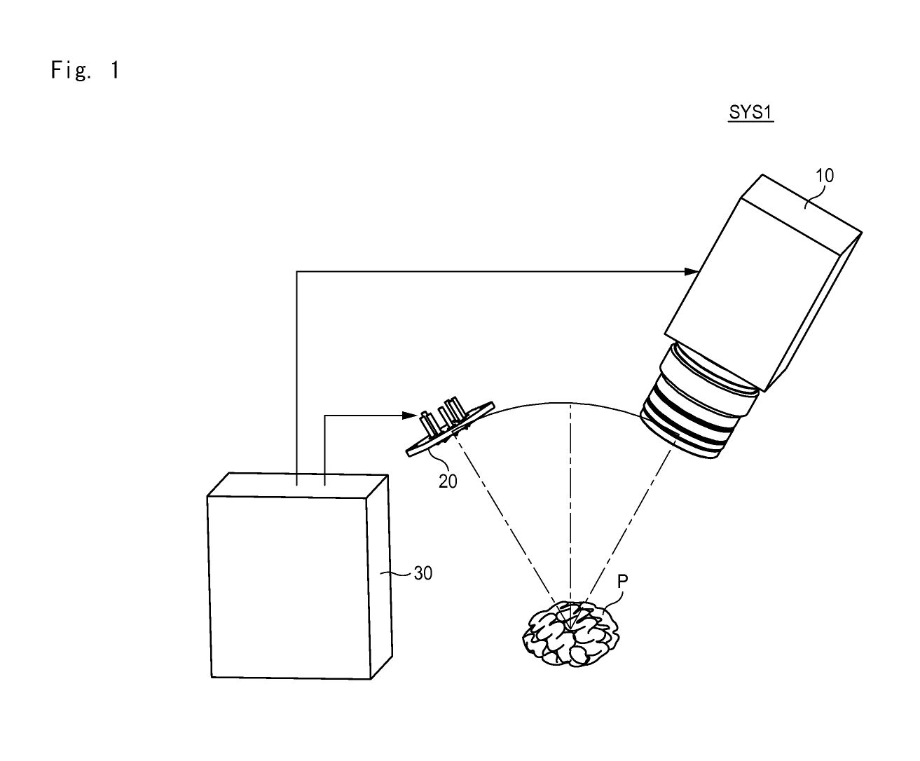

An imaging system according to a first embodiment will be described. FIG. 1 is a diagram showing an example of the imaging system according to the first embodiment. As shown in FIG. 1, an imaging system SYS1 includes an infrared camera 10, a lighting unit 20, and a control unit 30. The infrared camera 10 is a camera that is sensitive to light beams having wavelengths in an infrared region and is disposed so as to look into a subject P. In the present embodiment, for example, an InGaAs infrared camera 10 that is sensitive to wavelengths of up to 1.6 μm is used as an image sensor (infrared detector).

To provide an infrared camera that is sensitive to wavelengths of 1 μm or more, it is necessary to package a silicon readout IC and an infrared photodetector array with high density. For this reason, the number of effective pixels is smaller than that of a typical silicon image sensor in the price zone which can be used for medical purposes, and is currently a VGA class (640×524 pixels) at most. A typical CCD camera or CMOS camera is also sensitive to a near-infrared region. Instead of an InGaAs infrared camera, an InSb infrared camera (e.g., sensitive to wavelengths of 1.5 to 5 μm), amorphous Si microbolometer (e.g., sensitive to wavelengths of 7 to 14 μm), or the like may be used as an image sensor. However, the SN ratio of a mid-infrared camera sensitive to wavelengths of up to 2.5 μm degrades by about 100 times. For this reason, the following use form is conceivable: the near- and mid-infrared camera 10, which is sensitive to wavelengths of up 1.6 μm, is left as it is; and an additional camera for long wavelengths is disposed when the detection wavelength range is extended.

The infrared camera 10 includes an image sensor, as well as an imaging optical system (not shown). This imaging optical system includes a zoom lens for setting the imaging magnification by which the subject P is magnified and a focus lens for focusing on the subject P. The infrared camera 10 also includes a lens drive system (not shown) for driving one or both of the zoom lens and focus lens. The infrared camera 10 also includes a trigger input circuit or an interface synchronizable with IEEE1394 or the like.

The lighting unit 20 applies a light beam to the subject P. While, in FIG. 1, the incidence angle of a light beam emitted from the lighting unit 20 is the same as the angle at which the infrared camera 10 looks into the subject P, other incidence angles maybe used. The lighting unit 20 emits light beams having multiple wavelengths in an infrared region in a range including the wavelengths to which the infrared camera 10 is sensitive.

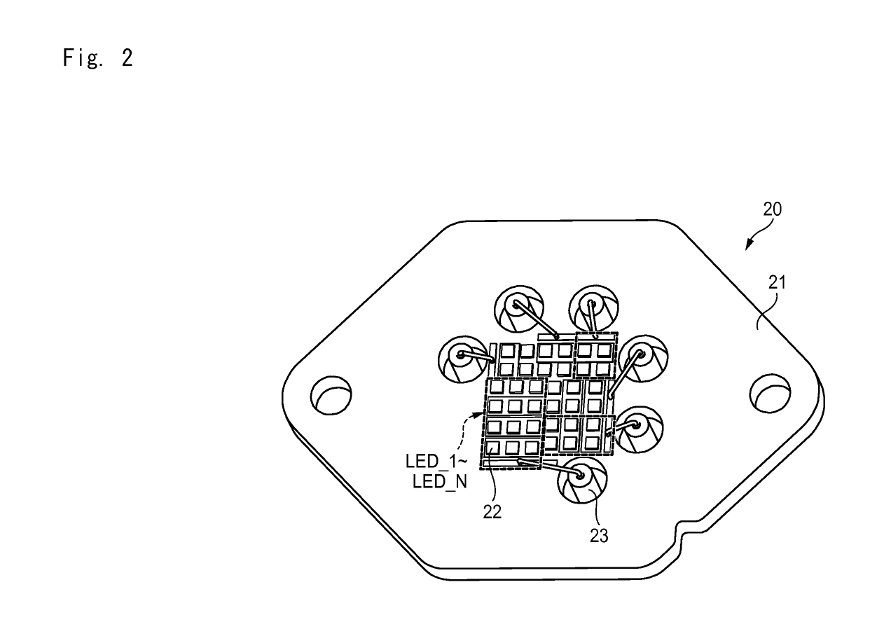

FIG. 2 is a perspective view showing an example of the lighting unit 20. The lighting unit 20 is an infrared light emitting diode (LED) module. As shown in FIG. 2, the lighting unit 20 includes a single metal package 21 and LEDs 22 that are mounted on the metal package 21 and emit infrared and visible light beams having several different wavelengths. In the present embodiment, the LEDs 22 emit six wavelengths, that is, 780, 850, 1050, 1200, 1330, and 1500 nm, respectively. The LEDs 22 are electrically connected to metal terminals 23. The LEDs 22 produce different light outputs due to the wavelengths thereof. Accordingly, in order to make light outputs corresponding to the respective wavelengths uniform, the number of mounted LEDs 22 is adjusted for each wavelength, or the number of serial or parallel connected LEDs 22 is adjusted for each wavelength, as shown by dotted lines in FIG. 2. Groups of LEDs 22 corresponding to the respective wavelengths are referred to as LED modules LED_1 to LED_N.

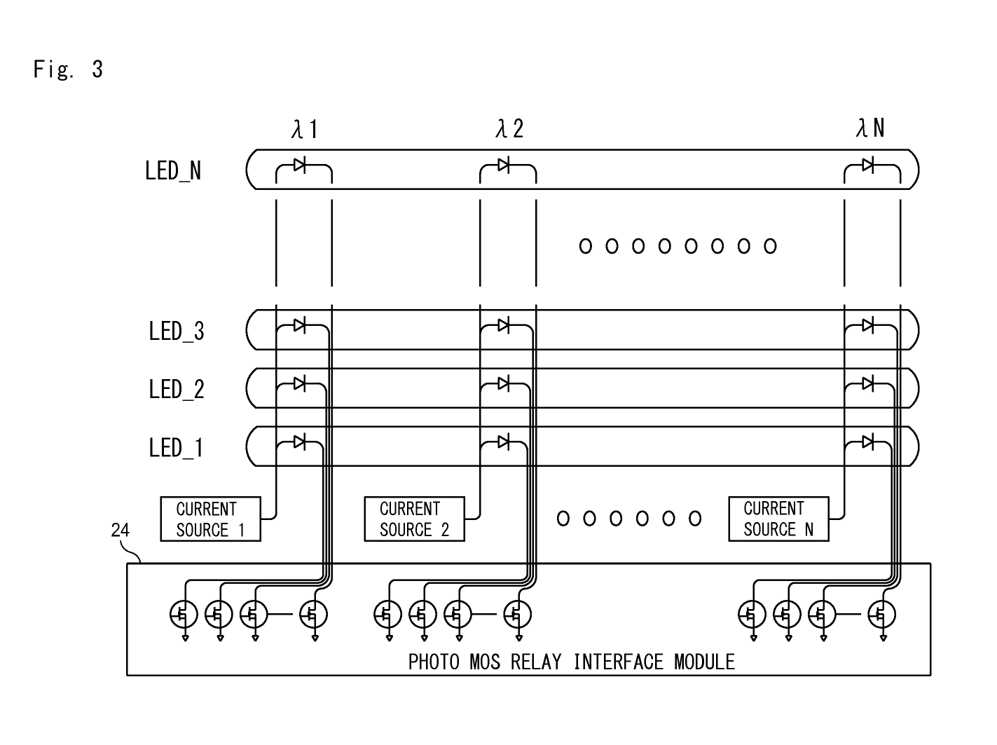

FIG. 3 is a diagram showing an example of a drive circuit of the lighting unit 20. As shown in FIG. 3, this drive circuit includes multiple current sources 1 to N corresponding to the LED modules LED_1 to LED_N and a photo MOS relay interface module 24 using metal-oxide-semiconductor field-effect transistors (MOSFETs). Although not shown in FIG. 3, the interface module 24 is connected to the bus of a personal computer (PC) and can turn or off any photo MOSFET in accordance with a program.

Since the terminal voltage of each LED 22 or the amount of output light thereof varies with the emission wavelength, the current sources 1 to N are disposed for the respective emission wavelengths. The photo MOS relay interface module 24 including a photo MOS relay switches between the LEDs 22 using a digital output circuit. The LED modules (LED_1 to LED_N) are groups of LEDs 22 having several different wavelengths from visible to infrared wavelengths. An LED module having a particular wavelength is connected to a single photo MOSFET. Turning on any photo MOSFET allows a particular LED module to be lighted, thereby allowing an infrared or visible light beam having a particular wavelength to be emitted. Further, turning on multiple photo MOSFETs simultaneously allows multiple wavelengths to be lighted.

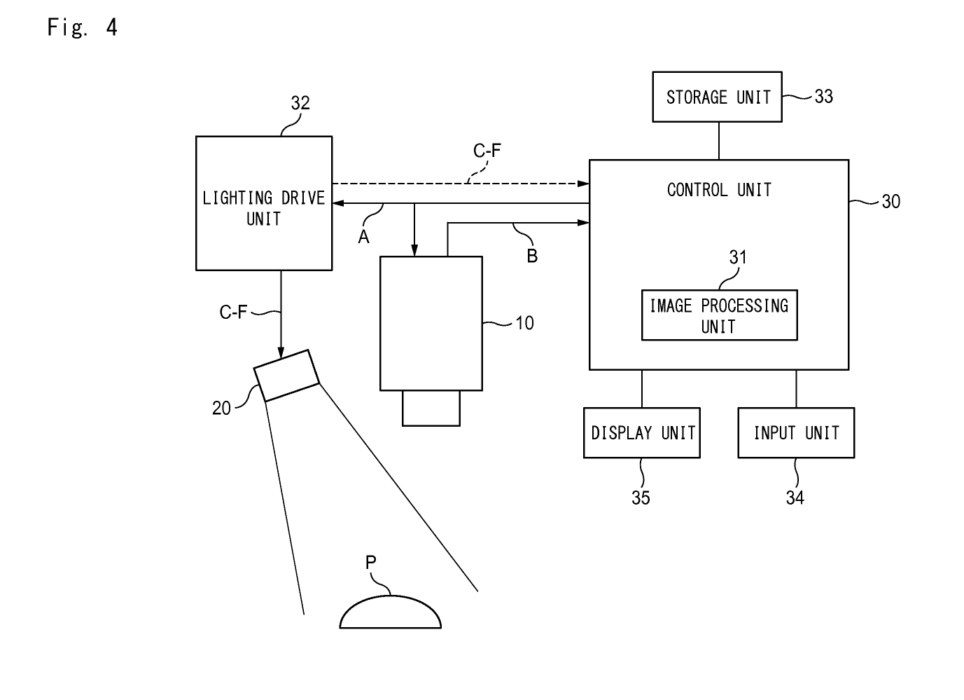

FIG. 4 is a function block diagram showing the imaging system SYS1. The control unit 30 includes an image processing unit 31, a lighting drive unit 32, a storage unit 33, an input unit 34, and a display unit 35. The control unit 30 is electrically connected to the infrared camera 10, as well as electrically connected to the lighting unit 20 through the lighting drive unit 32. The control unit includes an arithmetic processing unit, such as a central processing unit (CPU). This CPU controls the image processing unit 31 and the like on the basis of a control program stored in a storage unit (not shown), such as a hard disk. The control unit 30 generates a trigger signal A to be transmitted to the infrared camera 10 or lighting drive unit 32.

The image processing unit 31 processes an image signal transmitted from the infrared camera 10. The image processing unit 31 adjusts the color, contrast, or the like of the captured image, as well as combines multiple images. Combination of images includes combination of images having the same or different wavelengths, as well as generation of a stereoscopic image from multiple images as described later. The image processing unit 31 also generates a still or moving image from an image signal transmitted from the infrared camera 10.

The lighting drive unit 32 includes a drive circuit shown in FIG. 3 and lights one or more of LED modules (LED_1 to LED_N) specified by the control unit 30 on the basis of the trigger signal A transmitted from the control unit 30.

The storage unit 33 stores various programs, as well as stores the images processed by the image processing unit 31. The storage unit 33 includes an input/output (IO) device that can accommodate a storage medium, such as a hard disk, optical disk, CD-ROM, DVD-ROM, USB memory, or SD card.

A keyboard, a touchscreen, a pointing device such as a joystick or mouse, or the like is used as the input unit 34. When the input unit 34 is a touchscreen, the touchscreen may be formed on the display unit 35 (to be discussed later) so that an image displayed on the display unit 35 can be touched. By operating the input unit 34, the user performs, for example, the following: selection of the wavelength to be emitted from the lighting unit 20; setting of the imaging magnification of the infrared camera 10; focusing of the infrared camera 10; capture of an image; and storage of the images processed by the image processing unit 31 in the storage unit 33.

A liquid crystal display device, organic EL device, or the like is used as the display unit 35. The display unit 35 displays an image of the subject P captured by the infrared camera 10. The number of display units 35 need not be one, and multiple display units 35 may display images. A single display screen may display multiple images. In this case, one image may be a moving image, and the other images may be still images.

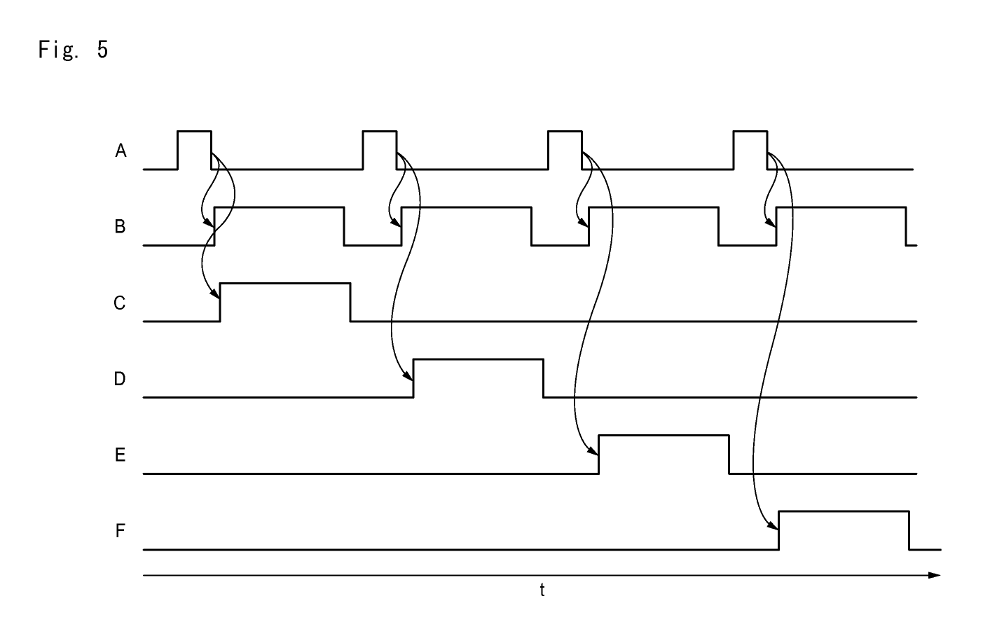

FIG. 5 is a diagram showing an example of an operation sequence of the imaging system SYS1. As shown in FIG. 5, upon receipt of the trigger signal A, the infrared camera 10 outputs an image signal B corresponding to one screen (one frame) to the control unit 30. The image signal B may be any of a single analog signal and a digital signal composed of multiple signal lines. The trigger signal A is also transmitted to the lighting drive unit 32 simultaneously.

As shown in FIG. 5, upon receipt of the trigger signal A, the lighting drive unit 32 sequentially outputs LED drive signals C to F corresponding to images (frames) to be captured. Thus, images corresponding to respective wavelengths captured by the infrared camera 10 are sequentially transmitted to the control unit 30 without being disturbed. For example, when the response speed of the photo MOSFET of the lighting drive unit 32 is 2 msec; the frame rate of the infrared camera 10 is 30 fps; and one frame is 1/30 second, the infrared camera 10 captures 30 infrared images having different wavelengths per second.

By always outputting LED drive signals, starting with an LED drive signal C, as described above, the control unit 30 can process images without erroneously combining the images and LED wavelengths. Alternatively, as shown by a broken line in FIG. 4, LED drive signals C to F may be simultaneously transmitted to the control unit 30 so that the LED drive signals C to F are stored together with images.

The different numbers of images to be captured (the different numbers of frames) maybe set to respective wavelengths for reasons, including: the sensitivity of the infrared camera 10 varies among wavelengths; and the infrared absorption rate of the subject P varies among wavelengths. While one of LED drive signals C to F is switched to another every frame in FIG. 5, for example, the following manner is possible. That is, an LED drive signal C is outputted twice continuously so that images having the same wavelength corresponding to two frames are captured; then LED drive signals D and E are outputted once respectively so that an image corresponding to one frame is captured for each signal; and then an LED drive signal F is outputted three time continuously so that images having the same wavelength corresponding to three frames are captured. Note that the number of images to be captured for each wavelength may be programmed as necessary. In this case, the image processing unit 31 of the control unit 30 executes the program.

As seen above, according to the present embodiment, the lighting unit 20 applies light beams having different wavelengths in an infrared region to the subject P, and the infrared camera 10 captures images of the subject P. Further, the lighting drive unit 32 switches between the wavelengths on the basis of a trigger signal, and the infrared camera 10 captures an image corresponding to a particular wavelength in synchronization with the wavelength switch. Since the infrared camera 10 does not require a spectral function, it can avoid the degradation of the camera sensitivity caused by a reduction in light amount and thus can capture a bright image without having to use a large gain.

Conventionally, the function of identifying a living body sample is improved by emphasizing the contrast between in-vivo components in a particular wavelength band using a bandpass filter. However, this approach has difficulty in quickly switching between wavelengths and cannot necessarily ensure the simultaneity of images. According to the present embodiment, a wavelength is switched to another every frame. Thus, it is possible to capture images corresponding to multiple wavelengths while switching between the wavelengths of light beams within 1/30 to 1/100 second.

Further, a combination of emission wavelengths most suitable for to the subject P can be selected by independently driving the LED modules having multiple emission wavelengths. For example, by emitting a first wavelength of 1500 nm and a second wavelength of 1100 nm simultaneously or with a slight time difference, it is possible to combine images emphasized by the wavelengths, as well as to generate a contrast-emphasized image approximately in real time. Note that the images are combined by the image processing unit 31 of the control unit 30.

In the present embodiment, the lighting unit 20 emits light beams having multiple wavelengths of 800 nm or more and 2500 nm or less and thus the infrared camera 10 can reliably capture images. Note that a wavelength shorter than 800 nm disadvantageously would make it difficult for the infrared camera 10 to capture an image; a wavelength longer than 2500 nm disadvantageously would reduce the SN ratio and thus make it difficult for the infrared camera 10 to capture a sharp image even when it uses an image sensor corresponding to that wavelength.

In the present embodiment, wavelengths from the lighting unit 20 may be 1000 nm or more and 1600 nm or less. Thus, the infrared camera 10 can more reliably capture images. In particular, an InGaAs infrared camera has effective sensitivity to this wavelength range and can easily capture images having multiple wavelengths.

In the present embodiment, the control unit 30 includes the lighting drive unit 32, which causes the lighting unit 20 to emit light beams having different wavelengths sequentially or simultaneously. Thus, a wavelength can be switched to another quickly and reliably by using the lighting drive unit 32. Even when a wavelength is switched to another, the simultaneity of images of the subject P can be ensured. Further, by emitting multiple wavelengths simultaneously, an infrared image can be displayed shortly without a PC or the like having to perform an operation. Thus, it is possible to capture a much sharper infrared spectral image much more quickly than a conventional hyperspectral camera provided with a dispersion spectrometer or FTIR spectrometer.

In the present embodiment, the control unit 30 synchronizes the switch between emission wavelengths by the lighting drive unit 32 and the capture of an image by the infrared camera 10. Thus, it is possible to accurately capture an image of the subject P for each wavelength, as well as to reliably associate the image with the wavelength with which the image has been captured.

In the present embodiment, the control unit 30 may assign a frame number to each wavelength in a manner corresponding to the degrees of the sensitivity of the infrared camera 10 to the wavelengths. Thus, when the sensitivity of the infrared camera 10 varies among wavelengths, an image corresponding to one wavelength to which the infrared camera 10 is less sensitive is avoided from becoming darker than images corresponding to the other wavelengths, for example, by capturing multiple images corresponding to that wavelength and then combining the images.

In the present embodiment, the control unit 30 includes the image processing unit 31, which combines images captured by the infrared camera 10. Thus, it is possible to combine multiple images corresponding to the same wavelength to generate a bright image, as well as to combine images corresponding to different wavelengths to generate a contrast-emphasized image.

When the LED modules (lighting unit 20) of the present embodiment apply light beams having wavelengths in the infrared region to the subject P such as a living body, a shallower region than that when using an X-ray is examined, since strong light dispersion occurs in the living body. Specifically, a relatively thin sample which lies within 1 to 2 cm from the living body surface or which is several cm or less thick is examined. Since the infrared camera 10 uses the optical lenses, the resolution is comparable to that when using an X-ray. Use of infrared light eliminates the possibility of radiation exposure, as well as may allow a lesion of a living body which cannot be trapped using an X ray to be detected with good sensitivity.

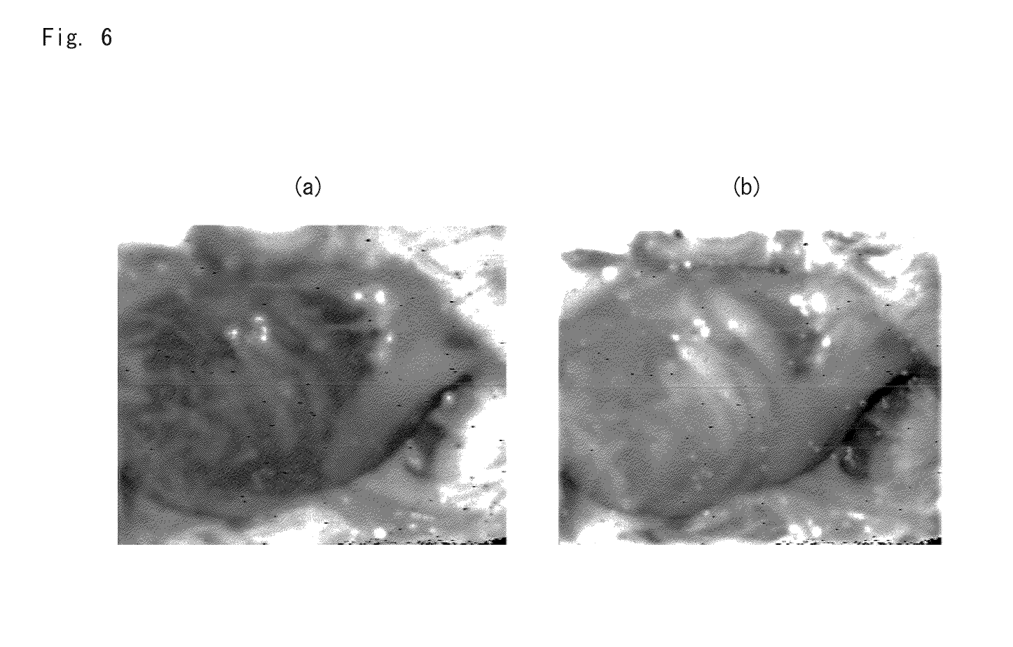

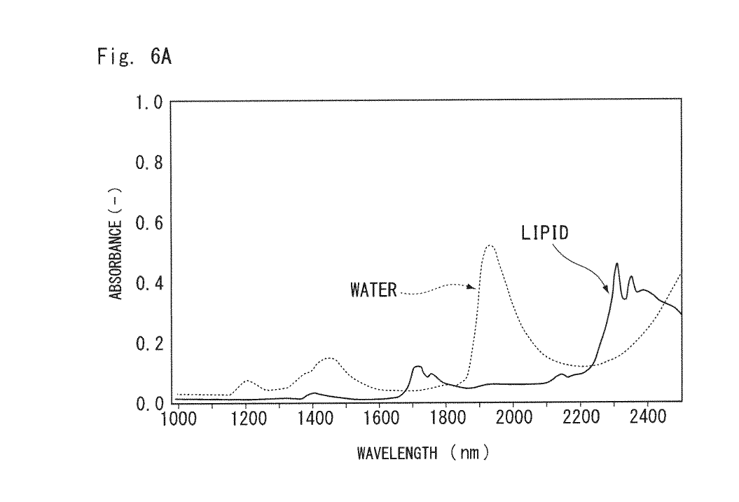

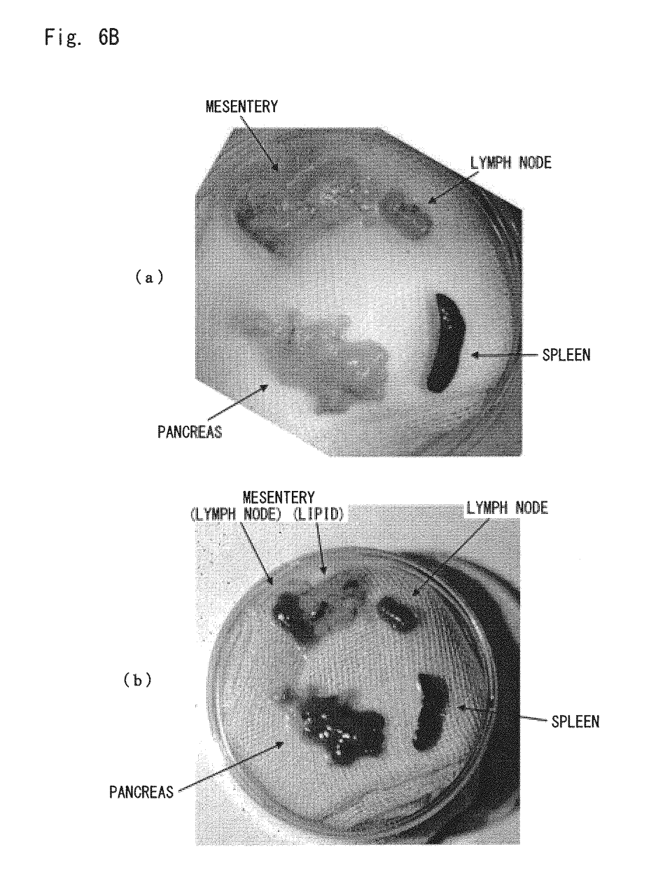

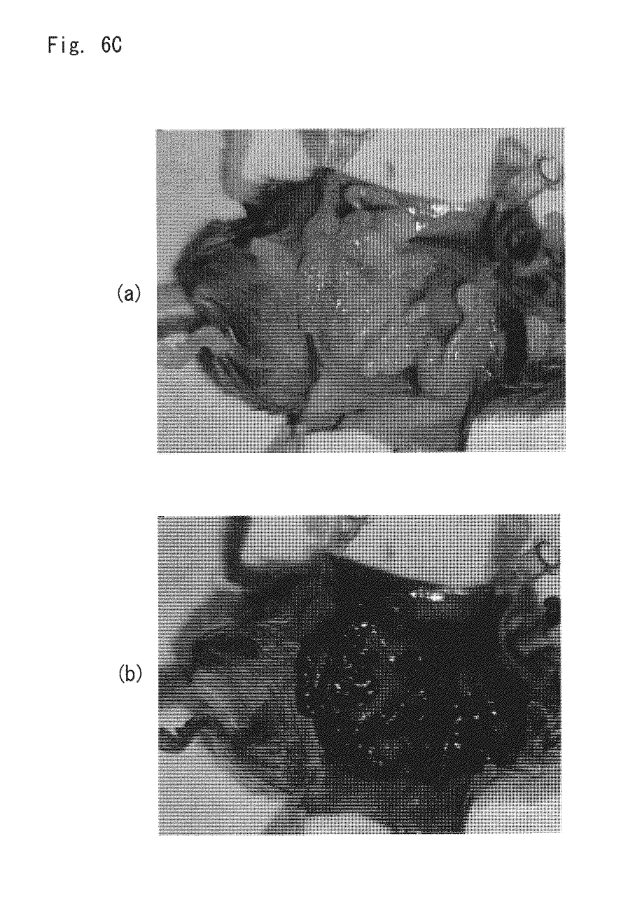

FIG. 6 includes drawings showing an example of an image captured by the imaging system SYS1. FIG. 6(a) shows an image of the abdomen of a mouse captured using LED light having a wavelength of 1550 nm, and FIG. 6(b) shows an image of the abdomen of the mouse captured using LED light having a wavelength of 1050 nm. An InGaAs infrared camera which was sensitive to wavelengths of up to 1.6 μm was used as the infrared camera 10. Both FIGS. 6(a) and 6(b) show images captured before the peritoneum was removed, and intraabdominal structures (organs) such as intestines can be visually recognized at infrared wavelengths of 1 μm or more. Further, an image indicating an organ could be obtained at a wavelength of 1550 nm. As seen above, use of light having a particular wavelength for illumination is an effective imaging technology which can significantly increase the amount of information about a living body.

FIG. 6A shows light absorption properties (spectral properties) of water and lipid in a near-infrared region. FIG. 6A indicates that water has higher absorbance than lipid at wavelengths of 1400 nm or more and 1600 nm or less and that both water and lipid have low absorbance at wavelengths shorter than 1200 nm. Further, for example, there are large differences in absorbance between water and lipid at wavelengths of 1200 to 1600 nm; there are small differences in absorbance between water and lipid at wavelengths shorter than 1200 nm. As seen above, water and lipid have the different degrees of absorbance with respect to near-infrared wavelengths, as light absorption properties (spectral properties) thereof in the near-infrared region (near-infrared wavelength region).

FIG. 6B(a) is a photograph taken by a visible light camera of living body tissues, such as pancreas, spleen, mesentery, and lymph node, removed from a mouse. Similarly, FIG. 6B(b) is a photograph of these living body tissues taken by an InGaAs infrared camera which is sensitive to wavelengths of up 1600 nm, using a light beam having a wavelength of 1600 nm emitted from an LED. Note that FIG. 6B(a) is a monochrome representation of a color image of the living body tissues captured by the visible light camera (to be discussed later) while applying a light beam having a wavelength of 400 to less than 800 nm to the living body tissues. The image captured by the visible light camera may be any of a color image and a monochrome image. In the image shown in FIG. 6B(a), only the spleen looks black under the visible light beam (e.g., a wavelength of 400 to less than 800 nm). In the image shown in FIG. 6B(b), on the other hand, the spleen, lymph node, and part of the mesenterium are highlighted in black. Instead of using the photograph taken by the infrared camera, the image of FIG. 6B(b) may be obtained, for example, by image synthesis including arithmetic processing such as obtaining the difference (e.g., the difference in light intensity) between the image captured by the infrared camera using a light beam having a wavelength of 1600 nm and the image captured by the visible light camera shown in FIG. 6B(a) serving as a reference image. Through this image synthesis, for example, it is possible to modify the shade, the color (black or the like) of the living body tissues, or the like to highlight portions absorbing the light beam having a wavelength of 1600 nm.

A histopathological examination revealed that the black position in the mesenterium shown in FIG. 6B(b) was a mesenteric lymph node. According to histological anatomy, 90% or more of cells forming mesenterium are fat cells and contain a large fat content, whereas pancreas and lymph node contain water including pancreatic juice as the main ingredient and water including lymph fluid as the main ingredient, respectively. Anatomically, pancreas is adjacent to mesenterium, and half or more thereof is embedded in retroperitoneum. The retroperitoneum tissue has approximately the same tissue composition as mesenterium and is a soft tissue including fat cells as the main ingredient. Accordingly, FIG. 6B(b), which is an observation result at a wavelength of around 1600 nm, can be obtained as an image in which the pancreas and lymph node are highlighted in black due to the absorbance of water. Further, it is possible to easily identify lymph node in mesenterium and pancreas adjacent to mesenterium and present in the fat tissue, which are difficult to identify under the visible light beam in FIG. 6B(a). Typically, the dissection of lymph node in the fat tissue is essential not only in performing an abdominal surgery for stomach cancer, colorectal cancer, pancreatic cancer, ovarian cancer, uterine cancer, or the like but also in performing a surgery for breast cancer or head and neck cancer. In this case, lymph node in the fat tissue can be accurately detected by referring to an image as shown in FIG. 6B(b). As a result, the risk of leaving behind lymph node due to an oversight can be reduced. Further, the boundary between pancreas and soft fat tissue is clarified and thus a solid organ such as pancreas can be treated safely.

FIGS. 6C(a) and 6C(b) are photographs taken by an InGaAs infrared camera which is sensitive to up 1600 nm and showing a mouse subjected to laparotomy. Specifically, FIG. 6C(a) is a photograph taken using a light beam having a wavelength of 1050 nm emitted from an LED, and FIG. 6C(b) is a photograph taken using a light beam having a wavelength of 1600 nm emitted from an LED. While many organs look bright in FIG. 6C(a), organs containing water are black and are easily distinguished from each other in FIG. 6C(b). Since the images shown in FIGS. 6C(a) and 6C(b) can be continuously captured by switching between the LEDs, the difference in light intensity between the images in FIGS. 6C(a) and 6C(b) can be easily obtained by processing using an electrical circuit, software, or the like. Thus, darkening or brightening of the whole image is prevented, and tissues containing much water, such as the fat tissue and lymph node, are easily distinguished from each other.

### Second Embodiment

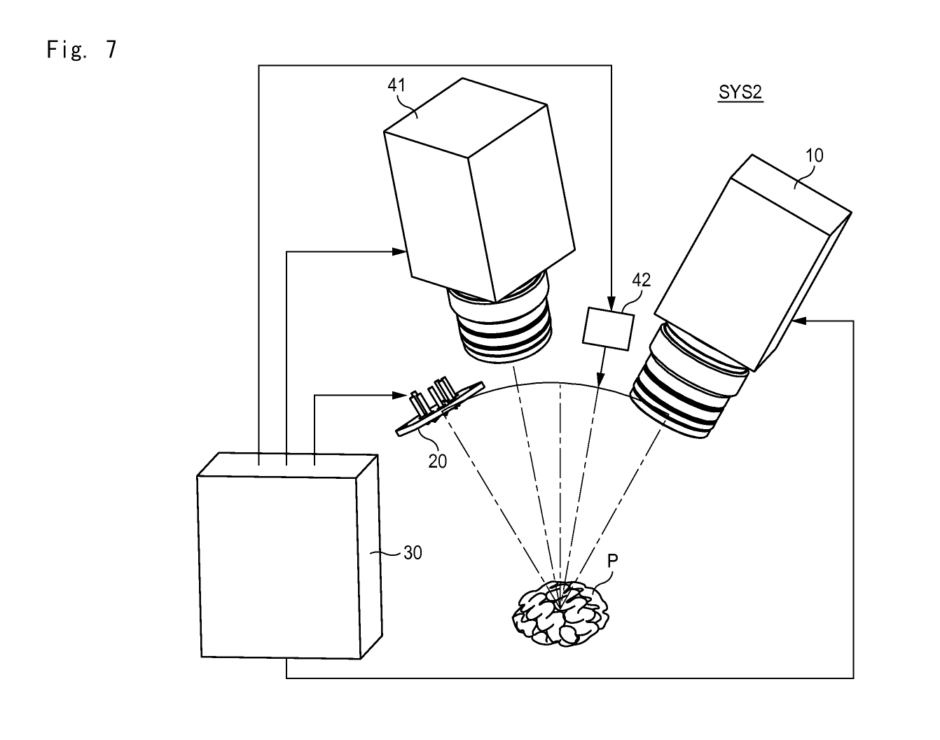

An imaging system according to a second embodiment will be described. In the present embodiment, elements which are the same as or similar to those in the above embodiment are given the same reference signs and the description thereof will be omitted or simplified. FIG. 7 is a diagram showing an example of the imaging system according to the second embodiment. As shown in FIG. 7, an imaging system SYS2 includes an infrared camera 10, a lighting unit 20, a control unit 30, a visible camera 41, and a visible lighting unit 42.

The visible camera 41 is a camera which is sensitive to light beams having wavelengths in the visible region. Light in the visible region is sensitive to the outer shape or surface shape of a subject P. A CCD camera or CMOS camera using a silicon image sensor capable of capturing an image of an outer shape or the like, such as a CCD image sensor or CMOS image sensor, is used as the visible camera 41. Note that a silicon image sensor can also capture images using a light beam having a wavelength of 1 μm or less. Accordingly, it is reasonable to use a visible camera 41 that captures images using a light beam having an infrared wavelength near the visible region, in terms of price and resolution.

The visible lighting unit 42 emits a light beam in the visible region. An LED lamp, as well as a laser light source, a halogen lamp, and the like are used as the visible lighting unit 42. Since the lighting unit 20 shown in FIG. 2 includes, as the LEDs 22, LED modules that emit visible light beams, the lighting unit 20 may be used as the visible lighting unit 42. The single lighting unit 20 may be used as a lighting unit for both the infrared camera 10 and visible camera 41.

The infrared camera 10 and visible camera 41 may capture images at different timings or at the same timing. Specifically, the infrared camera 10 and visible camera 41 may capture images in any of the following manners: the lighting unit 20 emits an infrared light beam and the infrared camera 10 captures an image of the subject P and, at a different timing, the visible lighting unit 42 emits a visible light beam and the visible camera 41 captures an image of the subject P; and the lighting unit 20 and visible lighting unit 42 emit an infrared light beam and a visible light beam, respectively, and the infrared camera 10 and visible camera 41 capture images simultaneously.

As seen above, according to the present embodiment, the visible lighting unit 42 emits a visible light beam (e.g., with a wavelength of 800 nm or less) which is sensitive to the outer shape or surface shape of the subject P and is relatively short, the lighting unit 20 emits an infrared light beam (e.g., with a wavelength of 1500 nm or less) which accommodates a deep structure or particular component of the subject P, and the visible camera 41 and the infrared camera 10 capture images using the respective light beams. Thus, it is possible to simultaneously obtain the outline and composition/component information about the living body (subject P) to shortly obtain an image with good visibility.

Assuming that the imaging system SYS2 is used as a surgery support system, by keeping the visible lighting unit 42 lighted and turning on and off only the infrared light LED modules of the lighting unit 20 in conjunction with the infrared camera 10, an infrared image can be displayed in real time without the operator losing visibility and with the thermal effect on the human body suppressed.

In the present embodiment, an imaging system for capturing an image of a living body tissue includes a lighting unit that emits infrared light beams having wavelengths in an infrared region based on spectral properties of water and lipid, an infrared camera that receives the infrared light beams, a visible lighting unit that emits a visible light beam having a wavelength in a visible region, a visible camera that receives the visible light beam, and a control unit including an image processing unit that processes an infrared image captured by the infrared camera using a visible image captured by the visible camera.

The infrared camera is sensitive to, for example, infrared light beams in a wavelength region of 800 nm or more and 2500 nm or less or infrared light beams in a wavelength region of 1000 nm or more and 1600 nm or less. The lighting unit emits, for example, infrared light beams having predetermined wavelengths in a wavelength region of 800 nm or more and 2500 nm or less, or infrared light beams having predetermined wavelengths in a wavelength region of 1000 nm or more and 1600 nm or less. The predetermined wavelengths may be, for example, wavelengths in a narrow band (e.g., wavelengths having a spectrum half-width of several nm or wavelengths of several tens of nm). The control unit includes a lighting drive unit that allows an infrared light beam and a visible light beam to be emitted sequentially or simultaneously. A visible image captured by the visible camera is, for example, an image as shown in FIG. 6B(a), and an infrared image captured by the infrared camera is, for example, an image as shown in FIG. 6B(b). The image processing unit of the control unit processes the infrared image using the visible image. The image processing may be image synthesis such as obtaining the difference between the infrared image and the visible image serving as a reference image, as described above, or may be other types of image processing. As seen above, the imaging system according to the present embodiment captures images of a living body tissue using at least two light beams (e.g., two infrared light beams, or an infrared light beam and a visible light beam) having predetermined wavelengths specified on the basis of the spectral properties of water and lipid. Thus, an image having high visibility can be obtained shortly and easily.

### Third Embodiment

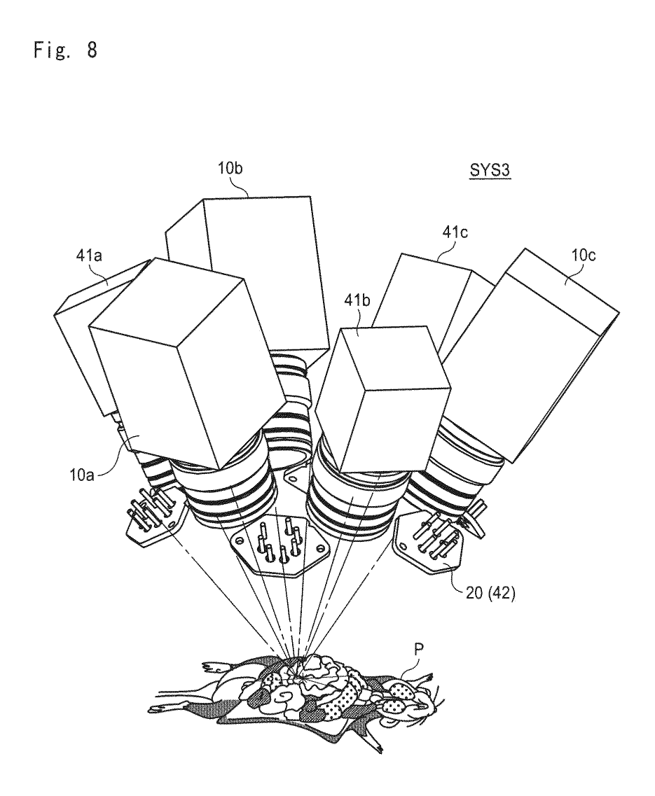

An imaging system according to a third embodiment will be described. In the present embodiment, elements which are the same as or similar to those in the above embodiments are given the same reference signs and the description thereof will be omitted or simplified. FIG. 8 is a diagram showing an example of the imaging system according to the third embodiment. As shown in FIG. 8, an imaging system SYS3 includes three infrared cameras, 10a to 10c, lighting units 20, and three visible cameras, 41a to 41c. Note that in FIG. 8, a control unit 30 is not shown. The lighting units 20 are used as visible lighting units 42. A subject P indicates a mouse subjected to laparotomy.

As shown in FIG. 8, the three infrared cameras, 10a to 10c, are disposed so as to look into a subject P at different angles. The infrared cameras 10a to 10c are also disposed in such a manner that the fields of vision thereof overlap each other on a flat or curved plane. While the infrared cameras 10a to 10c are of the same type, different types of infrared cameras 10 may be used. The number of infrared cameras need not be 3, and 2 or 4 or more infrared cameras may be disposed.

Lighting units 20 are disposed so as to correspond to the infrared cameras 10a to 10c. However, the lighting units 20 need not necessarily be disposed so as to correspond to the infrared cameras 10a to 10c, and a single lighting unit 20 may correspond to the infrared cameras 10a to 10c.

As with the infrared cameras 10a to 10c, the three visible cameras, 41a to 41c, are disposed so as to look into the subject P at different angles. While the visible cameras 41a to 41c are of the same type, different types of visible cameras 41 may be used. The number of visible cameras need not be 3, and 2 or 4 or more visible cameras may be disposed. Note that in the imaging system SYS3, the visible cameras 41a to 41c are disposed optionally.

Lighting units 20 are disposed as visible cameras 41 so as to correspond to the visible cameras 41a to 41c. However, the lighting units 20 need not necessarily be disposed so as to correspond to the visible cameras 41a to 41c, and a single lighting unit 20 may correspond to the visible cameras 41a to 41c, or the lighting units 20 corresponding to the infrared cameras 10a to 10c may also serve as those for the visible cameras 41a to 41c.

As shown in FIG. 8, the visible cameras 41a to 41c are disposed between the infrared cameras 10a to 10c. Accordingly, the interval at which the three infrared cameras, 10a to 10c, look into the subject P and the interval at which the three visible cameras, 41a to 41c, look into the subject P are approximately the same. However, the infrared cameras 10a to 10c and the visible cameras 41a to 41c need not be disposed as described above. For example, the visible cameras 41a to 41c may be disposed in positions remote from the infrared cameras 10a to 10c. Further, the number of infrared cameras and the number of visible cameras need not be the same. For example, the number of visible cameras may be smaller than the number of infrared cameras.

In the imaging system SYS3 shown in FIG. 8, the three infrared cameras, 10a to 10c, and lighting units 20 are disposed spatially. Thus, a stereoscopic infrared reflection image is obtained. Typically, analyzing the internal structure of the subject P from images captured by the infrared cameras 10a to 10c requires an image reconstruction algorithm for optical tomographic imaging. According to the imaging system SYS3, an image of an infrared reflection light beam from the subject P is captured, and a shape model and position thereof are limited to the shape or composition range within about 1 cm from the surface layer. Thus, it is possible to shortly recognize or display a lesion or tissue shape which is not exposed on the surface.

The imaging system SYS3 aims to identify not only the surface of a living body tissue but also a legion in a somewhat deep portion of the living body by using infrared light. For that purpose, the lighting units 20 are disposed so as to correspond to the infrared cameras 10a to 10c and visible cameras 41a to 41c; infrared and visible images are captured while changing the emission wavelength and position; and the images are analyzed by a computer. In an optical tomograph, many optical detectors obtain optical dispersion with respect to a point light source and transmission matrixes to identify a living body tissue. On the other hand, the imaging system SYS3 uses the disposed spatially multiple cameras in place of optical detectors.

By disposing the lighting units 20 spatially in advance, the light source position and emission wavelength can be swept without having to mechanically drive the lighting units. When the response speed of the photo MOSFET of the present embodiment (see the interface module 24 in FIG. 3) is, for example, 2 msec and the frame rate of the infrared cameras 10a to 10c during image capture is, for example, 30 fps (1 frame: 1/30 second), the infrared cameras 10a to 10c can each capture infrared images having different emission positions and wavelengths per second.

According to the present embodiment, there is provided the system in which the infrared cameras 10a to 10c, visible cameras 41a to 41c, and lighting units 20 are disposed spatially and which identifies a structure or legion inside a living body. Typically, an object can be stereoscopically recognized from the parallaxes of images captured by two or more cameras. In the present embodiment, the image processing unit 31 of the control unit 30 combines images from the visible cameras 41a to 41c to generate a stereoscopic image of the surface of a living body tissue serving as the subject P. The image processing unit 31 also combines images from the infrared cameras 10a to 10c to generate a stereoscopic image of the inside of the living body.

Further, by combining the stereoscopic image of the surface of the living body tissue and the stereoscopic image of the inside of the living body, the image processing unit 31 can generate a stereoscopic image in which the tissue surface and inside of the living body are combined. By displaying this stereoscopic image on the display unit 35, the user can simultaneously visually recognize the surface and inside of the living body serving as the subject P and thus can easily identify the internal shape corresponding to the surface of the subject P.

In the present embodiment, the multiple (preferably three or more) infrared cameras, 10a to 10c, and the multiple visible cameras, 41a to 41c, are disposed three-dimensionally. Accordingly, the infrared cameras 10a to 10c and visible cameras 41a to 41c have different parallaxes. For this reason, images captured by the infrared cameras 10a to 10c and visible cameras 41a to 41c may be modified on the basis of each other. Note that the images are modified by the image processing unit 31 of the control unit 30.

As seen above, according to the present embodiment, the multiple infrared cameras, 10a to 10c, look into the subject P at different angles. Thus, images of the subject P can be precisely observed at different angles. Further, by combining images in different fields of view, a stereoscopic image of the subject P can be generated. Further, by combining stereoscopic images obtained by the visible cameras 41a to 41c, the surface and inside of the subject P can be easily examined.

### Fourth Embodiment

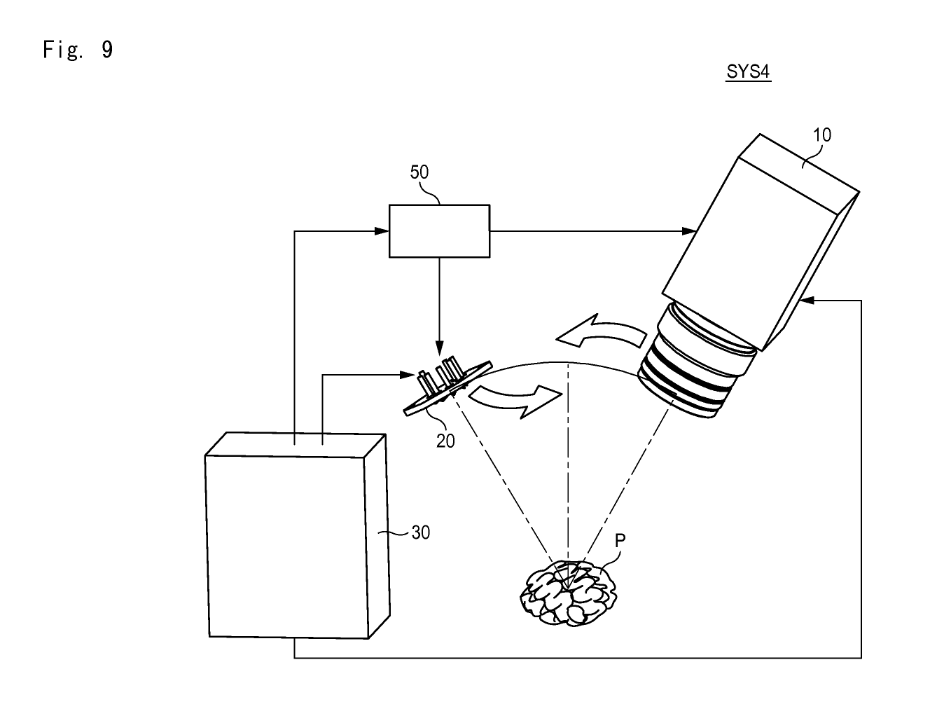

An imaging system according to a fourth embodiment will be described. In the present embodiment, elements which are the same as or similar to those in the above embodiments are given the same reference signs and the description thereof will be omitted or simplified. FIG. 9 is a diagram showing an example of the imaging system according to the fourth embodiment. As shown in FIG. 9, an imaging system SYS4 includes an infrared camera 10, a lighting unit 20, a control unit 30, and a drive unit 50.

As shown in FIG. 9, on the basis of an instruction from the control unit 30, the drive unit 50 moves the infrared camera 10 and lighting unit 20 so that the infrared camera 10 looks into an identical portion of a subject P in different fields of view. As the drive unit 50, a rotating motor, linear motor, or the like may move the infrared camera 10 and the like along a guide, or a robot arm or the like may be used. While the drive unit 50 moves the infrared camera 10 and lighting unit 20 together, it may move them separately. While FIG. 9 shows a rotation direction with the vertical direction of the subject P as the central axis, as the direction in which the drive unit 50 moves the infrared camera 10 and the like, any direction such as the vertical direction or spiral direction may be set.

The control unit 30 instructs the drive unit 50 to move the infrared camera 10 and the like, as well as causes the infrared camera 10 to capture images of the subject P in multiple movement positions. The lighting unit 20 emits an infrared light beam having a predetermined wavelength at the timing when the infrared camera 10 captures an image. Thus, the infrared camera 10 can capture multiple images of the subject P at different angles. The adjustment of the imaging magnification of the infrared camera 10 or focusing thereof is performed with the movement of the infrared camera 10 as necessary. The infrared camera 10 and the like may be moved in a preprogrammed direction, speed, or the like, or the user may manually move them using the input unit 34 such as a joystick.

The imaging system SYS4 maybe provided with the visible camera 41 and visible lighting unit 42 shown in FIG. 7. The visible camera 41 and visible lighting unit 42 may be moved together with the infrared camera 10 or separately therefrom by the drive unit 50.

As seen above, according to the present embodiment, the infrared camera 10 and the like are moved by the drive unit 50. Thus, the infrared camera 10 can easily capture multiple images of the subject Pat different angles. Further, since the infrared camera 10 is moved, the number of disposed infrared cameras 10 can be reduced and thus the system cost can be reduced.

### Fifth Embodiment

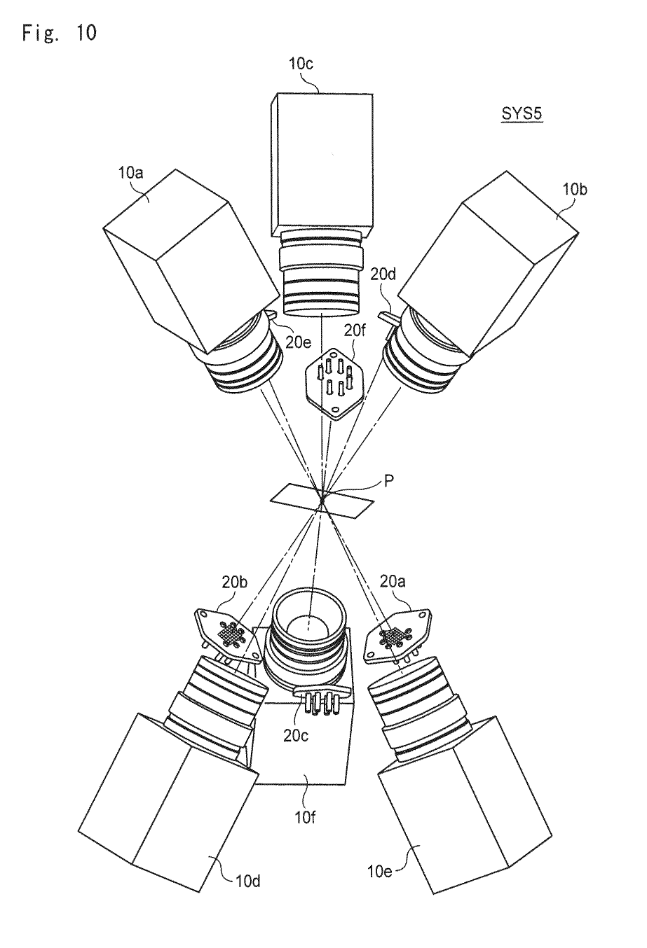

An imaging system according to a fifth embodiment will be described. In the present embodiment, elements which are the same as or similar to those in the above embodiments are given the same reference signs and the description thereof will be omitted or simplified. FIG. 10 is a diagram showing an example of the imaging system according to the fifth embodiment. As shown in FIG. 10, an imaging system SYS5 includes six infrared cameras, 10a to 10f, and lighting units 20. Note that a control unit 30 is not shown.

In the imaging system SYS5, infrared cameras 10d and the like and lighting units 20 are additionally disposed below a subject P. Thus, images of light beams transmitted through the subject P are captured. Images of small animals or most of surgically resected samples (surgical pathological samples) having a thickness of 3 to 4 cm or less can be captured by the high-sensitivity infrared cameras 10a to 10e by using transmitted light beams. Transmitted infrared light beams can be sufficiently detected by the infrared cameras 10a to 10e by preventing the light beams from directly entering the infrared cameras 10a to 10e.

As shown in FIG. 10, in the imaging system SYS5, the three infrared cameras, 10a to 10c, are disposed in front of the subject P, and the three infrared cameras, 10d to 10f, are disposed behind the subject P. However, the same number of infrared cameras 10 need not be disposed both in front of and behind the subject P. For example, a smaller number of infrared cameras 10 may be disposed behind the subject P than in front thereof. Lighting units 20a to 20f are disposed so as to correspond to the infrared cameras 10a to 10f and to sandwich the subject P.

In the imaging system SYS5, images of light beams transmitted through the subject P are captured by the infrared cameras 10a to 10f while sequentially switching between the lighting units 20a to 20f. The image processing unit 31 of the control unit 30 generates stereoscopic images corresponding to the front and back sides of the subject P by combining the images from the six infrared cameras, 10a to 10c. Thus, it is possible to construct a simple optical CT system without a mechanical drive mechanism.

The imaging system SYS4 may be provided with the visible camera 41 and visible lighting unit 42 shown in FIG. 7 in front of and/or behind the subject P. Note that a visible light beam from the visible lighting unit 42 is not transmitted through the subject P. Accordingly, the visible camera 41 is disposed so as to capture an image of a light beam reflected by the subject P.

As seen above, according to the present embodiment, images of light beams transmitted through the subject P are captured both in front of and behind the subject P. Thus, the internal structure of the subject P can be easily examined. Further, by combining the images from the infrared cameras 10a to 10f, it is possible to generate a stereoscopic image in which the front and back sides of the subject P are combined and thus to easily recognize the internal structure of the subject P.

### Sixth Embodiment

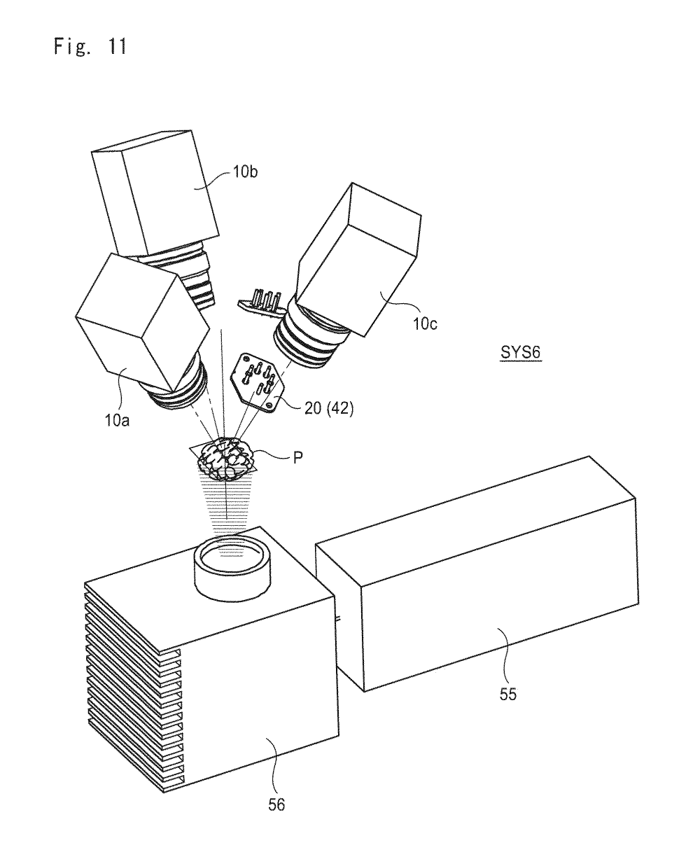

An imaging system according to a sixth embodiment will be described. In the present embodiment, elements which are the same as or similar to those in the above embodiments are given the same reference signs and the description thereof will be omitted or simplified. FIG. 11 is a diagram showing an example of the imaging system according to the sixth embodiment. As shown in FIG. 11, an imaging system SYS6 includes three infrared cameras, 10a to 10c, a lighting unit 20, an infrared laser 55, and a galvano scanner 56. Note that a control unit 30 is not shown.

The infrared laser 55 emits a line-shaped laser light beam having a predetermined wavelength in accordance with an instruction from the control unit 30. The galvano scanner 56 includes a galvano mirror (not shown) and sweeps the line-shaped laser light beam emitted from the infrared laser 55 in a predetermined direction. Note that the infrared laser 55 may emit a spot-shaped laser light beam for scanning.

In the imaging system SYS6, the infrared laser 55 and galvano scanner 56 are disposed below a subject (living body sample) P. Thus, a transmission measurement of the subject P is possible. As described above, it is possible to detect transmitted infrared light using the spatially disposed infrared cameras 10a and the like as necessary and thus to construct a simple optical CT system. In conventional stereoscopy, the surface shape of an object is recognized three-dimensionally. However, detecting a structure inside a living body sample requires further removing the redundancy of detection of congruent points.

In the imaging system SYS6, the galvano scanner 56 below the subject P sweeps a laser light beam from the infrared laser 55. A feature of the imaging system SYS6 is that three-dimensional congruent points are easily obtained. Before the infrared cameras 10a to 10c capture images, first, a semi-transparent film is placed at a particular height and then the application position of a laser light beam and the coordinates of the bright points of the infrared cameras 10a to 10c are calibrated. Thus, a stereoscopic structure in the subject P can be calculated from multiple images captured by the infrared cameras 10a to 10c on a section along a particular plane.

An optical CT is effective in knowing the active state of brain, or the like. However, an optical CT is formed by combining a discrete light emitting source and a discrete light receiving element and therefore the number of elements that provide information necessary to reconstruct an image is limited. For this reason, it does not provide sufficient resolution. In the imaging system SYS6, the infrared camera 10a is used as a light-receiving element, and line light generated by the infrared laser 55 and galvano scanner 56 is used as a light-emitting element. Thus, there is realized an optical CT that increases the number of elements equivalently and drastically improves resolution.

A light beam emitted from an LED or halogen lamp and then transmitted directly through the periphery of the subject (sample) P is too strong. For this reason, images captured by the infrared cameras 10a to 10c are disadvantageously saturated. On the other hand, by using the galvano scanner 56, the sweeping shape of a laser light beam can be freely set. Thus, it is possible to suppress light beams transmitted directly through the periphery of the subject P and thus to prevent the saturation of captured images. Further, by disposing a half mirror between the galvano scanner 56 and infrared laser 55 to detect the intensity of reflected light, the function of a confocal stereomicroscope can be provided.

The field of view may be expanded by mechanically sweeping a group of the infrared cameras 10a to 10c and lighting unit 20 as a whole. Note that effects similar to those obtained by mechanically driving them are obtained by disposing the infrared cameras 10a to 10c on a flat or curved plane in such a manner that the fields of view thereof overlap each other. As described above, images of small animals or surgical pathological samples having a thickness of 3 to 4 cm or less can be captured by the high-sensitivity infrared cameras 10a to 10c by using transmitted light beams. However, the light beams are attenuated by about several digits due to light absorption inside the living body and therefore free space light has to be blocked. For this reason, in the imaging system SYS6, an aperture corresponding to the size of the subject P may be formed in the base so that only light beams transmitted through the subject P enter the infrared cameras 10a to 10c. The imaging system SYS6 may be provided with the visible camera 41 and visible lighting unit 42 shown in FIG. 7.

As seen above, according to the present embodiment, images of transmitted light beams based on laser light beams swept by the galvano scanner 56 are captured by the infrared cameras 10a to 10c. Thus, the resolution of the captured images can be improved.

### Seventh Embodiment

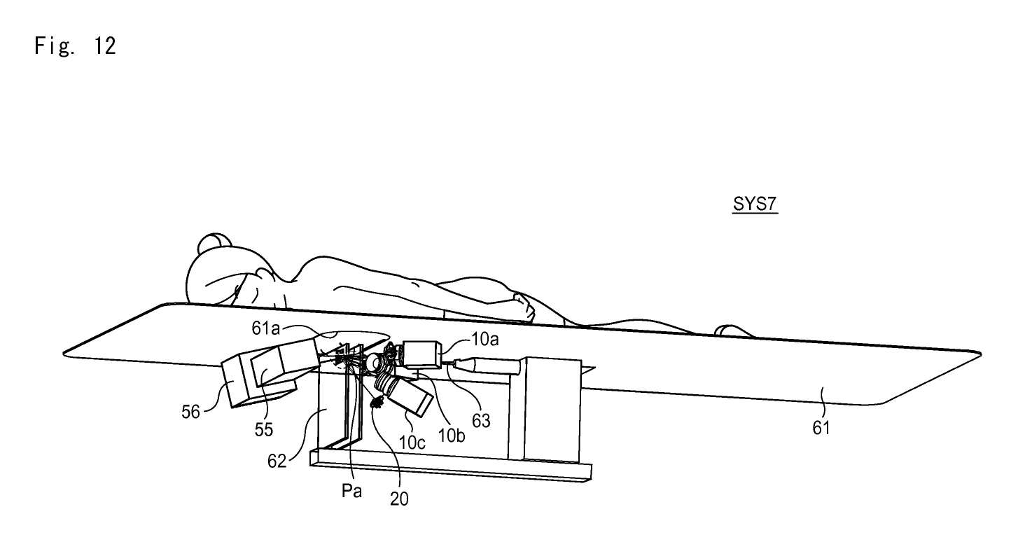

An imaging system according to a seventh embodiment will be described. In the present embodiment, elements which are the same as or similar to those in the above embodiments are given the same reference signs and the description thereof will be omitted or simplified. FIG. 12 is a diagram showing an example of the imaging system according to the seventh embodiment. In this example, an imaging system SYS7 is applied to a mammotome. As with the imaging system SYS6 shown in FIG. 11, the imaging system SYS7 includes three infrared cameras, 10a to 10c, lighting units 20, an infrared laser 55, and a galvano scanner 56. Note that a control unit 30 is not shown.

The imaging system SYS7 also includes a bed 61, a transparent plastic plate 62, and a perforation needle 63. The bed 61 is a bed on which an examinee lies with his or her face down and is formed so as to be thin. The bed 61 has an aperture 61a through which a breast Pa of the examinee serving as the subject is exposed downward. The transparent plastic plate 62 is used to sandwich both sides of the breast Pa to flatten it. The perforation needle 63 is inserted into the breast Pa in a core needle biopsy to take a sample.

The infrared cameras 10a to 10c, lighting units 20, infrared laser 55, and galvano scanner 56 are disposed below the bed 61. The infrared cameras 10a to 10c are disposed with the transparent plastic plate 62 between the infrared cameras 10a to 10c and galvano scanner 56. The infrared cameras 10a to 10c and lighting units 20 are disposed so as to form a spherical shape. The lighting units 20 may emit light beams having multiple wavelengths including at least one infrared wavelength of 1000 nm or more.

As shown in FIG. 12, the breast Pa is flattened by pressing the transparent plastic plate 62 against both sides thereof; in this state, the lighting unit 20 and infrared laser 55 sequentially emit infrared light beams having predetermined wavelengths; and the infrared cameras 10a to 10c capture images. More specifically, the infrared cameras 10a to 10c capture images of the breast Pa using reflected infrared light beams from the lighting units 20, as well as capture images of the breast Pa using transmitted infrared light beams based on laser light beams swept by the galvano scanner 56.

By overlapping the fields of view of the infrared cameras 10a to 10c and sequentially lighting the lighting units 20 and infrared laser 55 located in different positions, the inside of the breast Pa can be displayed as a stereoscopic image. Further, the stereoscopic shape of a legion can be grasped. Currently, two-dimensional and three-dimensional mammography using a digital X-ray image sensor is being widely used in breast cancer screening. However, even when infrared is used, a stereoscopic shape recognition function can be performed by combining the infrared cameras 10a to 10c and lighting units 20.

In a conventional core needle biopsy, a perforation needle (core needle) is inserted while measuring the depth of the needle using ultrasonic echo. The infrared mammotome in FIG. 12 determines the three-dimensional coordinates of a legion using a confocal stereomicroscope (including the infrared laser 55 and galvano scanner 56) and then inserts the perforation needle 63 into the breast Pa to take a sample.

As seen above, according to the present embodiment, the infrared mammotome using the difference between infrared spectrums is used in a core needle biopsy. Thus, a sample can be taken on the basis of the spatial recognition of an accurate tissue image. Further, imaging using infrared light, which does not cause X-ray exposure, has an advantage that it can be usually used in obstetrics and gynecology, regardless of whether the patient is pregnant.

While the imaging system SYS7 in FIG. 12 is applied to a mammotome, it can also be used as mammography by obtaining images of the inside of the breast Pa using the infrared cameras 10a to 10c.

### Eighth Embodiment

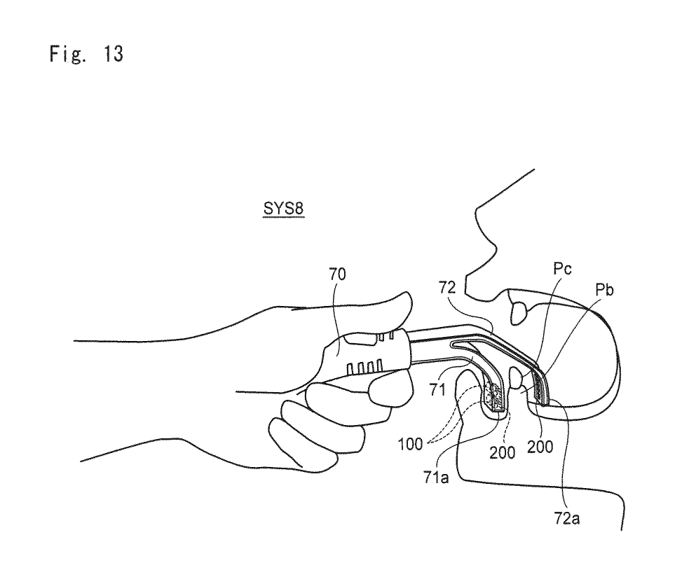

An imaging system according to an eighth embodiment will be described. FIG. 13 is a diagram showing an example of the imaging system according to the eighth embodiment. In this example, an imaging system SYS8 is applied to a tooth row imaging device. As shown in FIG. 13, the imaging system SYS8 includes abase 70, holding plates 71 and 72, infrared and visible LED chips (lighting units) 200, and visible expansion type small infrared cameras 100.

The base 70 is a part grasped by the user, robot hand, or the like. All or some components (lighting drive unit 32 and the like) of a control unit 30 may be housed in the base 70. When the control unit 30 and the like are housed in the base 70, the control unit 30 and the like are electrically connected to an external PC or display unit 35 by wire or wirelessly.

The holding plates 71 and 72 are formed by bifurcating a single member extending from one edge of the base 70 into two parts and bending the two parts in the same direction. The distance between an edge 71a of the holding plate 71 and an edge 72a of the holding plate 72 is set to a distance such that a gum Pb (to be discussed later) or the like can be located therebetween. The holding plates 71 and 72 may be formed of, for example, a deformable material so that the distance between the edges 71a and 72a can be changed.

The two small infrared cameras 100 are vertically disposed on a part opposite to the edge 72a of the holding plate 72, of the edge 71a of the holding plate 71. The small infrared cameras 100 are infrared cameras that can also capture images in the visible region. While the two small infrared cameras, 100a, are disposed in FIG. 13, one or three or more infrared cameras may be disposed. The disposition of the small infrared cameras 100 are optional. By disposing the multiple small infrared cameras 100 with parallaxes, a stereoscopic image can be generated. The small infrared cameras 100 and base 70 are electrically connected together through the inside of the holding plate 71.

The edges 71a and 72a of the holding plates 71 and 72 are provided with the LED chips 200. As with the lighting units 20, the LED chips 200 each include multiple LEDs that emit multiple wavelengths in the infrared region and a wavelength in the visible region. The LED chips 200 and base 70 are electrically connected together through the inside of the holding plates 71 and 72. The LED chips 200 may emit light beams having multiple wavelengths including at least one infrared wavelength of 1000 nm or more.

In the imaging system SYS8, the holding plates 71 and 72 are inserted into the mouse of an examinee, and a gum Pb or tooth Pc serving as the subject is disposed between the edges 71a and 72a thereof. Subsequently, the LED chips 200 at the edge 72a are driven, and images of the gum Pb or the like are captured by the small infrared cameras 100 while changing the infrared wavelength. Simultaneously, the LED chips 200 at the edge 71a emit visible light beams, and the small infrared cameras 100 capture images. Subsequently, the base 70 is moved to move the small infrared cameras 100 along the tooth row; the small infrared cameras 100 capture images for each of connectable fields of view as necessary; and the images are combined to obtain an entire image along the tooth row. The small infrared cameras 100 may be caused to make steps corresponding to predetermined distances and to capture an image at each step, or the small infrared cameras 100 may be caused to move at a predetermined speed and to capture images as necessary.

As seen above, according to the present embodiment, stereoscopic images in respective positions are automatically combined using software. Thus, a surface stereoscopic model of the gum Pb or tooth Pc obtained using visible light and pathological information about the inside of the gum can be obtained simultaneously. While a tooth row can be measured three-dimensionally using an X-ray CT, usual legions cannot be indiscriminately observed using an X-ray CT in dental services, which require a relatively strong X-ray. In this respect, infrared images are suitable for usual intraoral observation. As another feature, infrared images are sensitive to legions which change the distribution of blood or water, such as edema and irritation.

### Ninth Embodiment

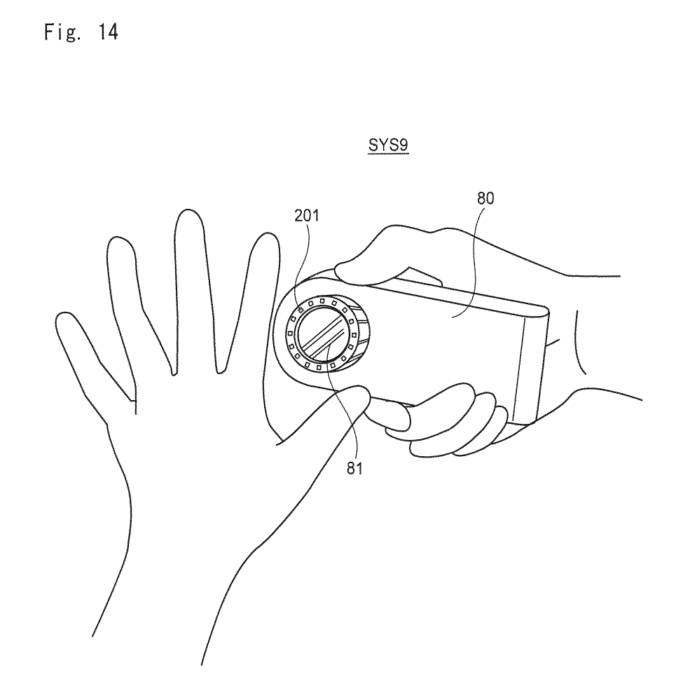

An imaging system according to a ninth embodiment will be described. FIG. 14 is a diagram showing an example of the imaging system according to the ninth embodiment. In this example, an imaging system SYS9 is applied to a dermoscope. As shown in FIG. 14, the imaging system SYS9 includes a body 80 having a shape that the user can hold with a hand. A visible and infrared camera is housed in the body 80, and an imaging lens thereof 81 is disposed in a part of the body 80.

Many infrared and visible LED chips (lighting units) 201 are concentrically fitted into the periphery of the imaging lens 81 and emit light beams having wavelengths from ultraviolet to infrared wavelengths. Images of the subject can be captured at multiple emission angles through the imaging lens 81 while changing the infrared wavelength using the LED chips 201. The images may be combined by an image processing unit in the body 80, or the image data may be transmitted to an external PC or the like so that the images are combined on the PC. Polarizing plates may be provided on the LED chips 201 and imaging lens 81 in such a manner that the polarization directions are perpendicular to each other and thus, for example, the reflection from the skin surface serving as the subject may be suppressed. The LED chips 201 may emit light beams having multiple wavelengths including at least one infrared wavelength of 1000 nm or more.

As seen above, according to the present embodiment, images of the surface and inside of the skin serving as the subject are obtained. Thus, it is possible to repeatedly and shortly obtain a surface model and pathological information about the inside and to easily perform a dermoscopy examination or the like.

### Tenth Embodiment

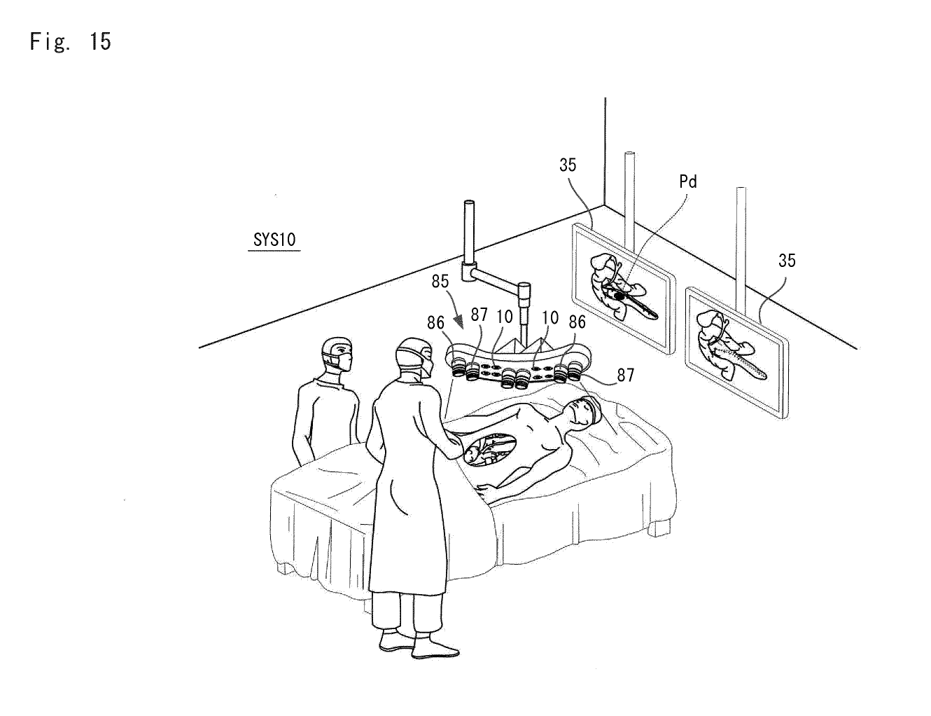

An imaging system according to a tenth embodiment will be described. In the following description, FIG. 15 is a diagram showing an example of the imaging system according to the tenth embodiment. In this example, an imaging system SYS10 is applied to an infrared imaging intraoperative support system. As shown in FIG. 15, the imaging system SYS10 includes an operation lamp 85 and two display units 35.

In the operation lamp 85, multiple infrared LED modules (lighting units), 87, and infrared cameras 10 are embedded between multiple visible lamps, 86, that emit visible light beams. In FIG. 15, three visible lamps 86, three infrared LED modules 87, and eight infrared cameras 10 form the operation lamp 85. Images captured by the infrared cameras 10 can be displayed on the display units 35. The two display units 35 may display the same image, or may display different images having different infrared wavelengths. In FIG. 15, the left display unit 35 is displaying an in-vivo cancer cell Pd. A visible camera 41 or the like (see FIG. 7) may be disposed on the operation lamp 85; an image may be captured by the visible camera using a visible light beam; and this image may be displayed on the display units 35.

As seen above, according to the present embodiment, infrared images having different wavelengths can be captured by the infrared cameras 10 using a visible light beam emitted from the visible light lamp 86 while switching between light beams having infrared wavelength using the infrared LED modules 87.

The invasiveness and efficiency of an operation or treatment are determined by the range and intensity of injury or cautery associated with incision and hemostasis. To prevent a surgical complication, it is important whether the operator can visually recognize a legion, as well as nerves, solid organs such as pancreas, fat tissue, blood vessels, and the like. While intelligent operating rooms have been proposed recently in which an X-ray CT or MRI device is disposed so as to be connected to an operating room so that intraoperative diagnosis can be made quickly, such facilities are expensive. Further, such facilities require a special environment and also require that an operation be suspended. The infrared imaging intraoperative support system according to the above embodiment, in which the multicolor (multi-wavelength) LED module 87 and infrared camera 10 are combined, can be realized, for example, by only making a modification such as embedding the LED module 87, infrared camera 10, and the like in an existing operation lamp. Further, since the visible lighting lamps can be lighted at all times, an operation is not obstructed.

While the present invention has been described using the first to tenth embodiments, the technical scope of the invention is not limited to the scope described in the embodiments. Various changes or modifications can be made to the embodiments without departing from the spirit and scope of the invention. One or more of the elements described in the embodiments may be omitted. Any forms resulting from such changes, modifications, or omission are included in the technical scope of the invention.

At least two of the first to tenth embodiments may be combined. For example, the drive unit 50 of the fourth embodiment maybe applied to the imaging system SYS3 of the third embodiment or the imaging system SYS5 of the fifth embodiment so that the infrared cameras 10a to 10c or visible cameras 41a to 41c are moved by the drive unit 50.

While LEDs are used as the lighting units 20 and the like in the first to tenth embodiments, laser light sources such as infrared lasers may be used in place of LEDs.

In the first to tenth embodiments, infrared light beams emitted from the lighting unit 20 and the like need not be coherent light beams having a single wavelength. For example, a light beam having a desired infrared wavelength as the center wavelength and having a predetermined wavelength width may be emitted. More specifically, a wavelength of 1050 nm or 1330 nm specified above may be emitted as a single wavelength, or a light beam having a wavelength of 1050 nm or 1330 nm as the central wavelength and having a predetermined wavelength width may be emitted.

The lighting unit emits light beams having multiple wavelengths including at least one infrared wavelength of 1000 nm or more and is mounted on at least one of a mammotome, mammography device, a dermoscope, and a tooth row transmission device. However, the lighting unit 20 or the like may be mounted on not only a mammotome or the like but also other types of devices. Further, the imaging system of the above embodiment can be applied to an intraoperative (operation) support system. For example, the imaging system can be used in combination with a treatment device (e.g., a device for incision, hemostasis, perforation, or the like with respect to a living body tissue) in an operation support system, or can be used as a device formed integrally with such a treatment device. Further, an operation support system may include the above imaging system and a treatment device for treating a living body tissue as described above.

In the first to tenth embodiments, the resolution may be improved or the influence of a defective pixel occurring in the infrared camera 10 or the like may be corrected by combining multiple images captured by one infrared camera 10 or the like while changing the field of view, or by combining images captured by multiple infrared cameras, 10, or the like.

In the first to tenth embodiments, image data captured using an infrared wavelength may be corrected on the basis of an image previously captured in a dark state in which light beams having any infrared wavelengths are not emitted or in a state in which outside light is naturally entering. For example, by obtaining the difference between an infrared image and an image captured in a dark state as described above and then combining images having respective wavelengths on a PC or the like, it is possible to obtain a recognition support image indicating the surface shape and internal composition of the subject P.

Some elements of the imaging systems SYS1 to SYS10 may be implemented by a computer. For example, the control unit 30 may be implemented by a computer. In this case, on the basis of a control program, the computer performs a process in which the lighting units 20 or the like apply light beams having multiple wavelengths in the infrared region to the subject P and a process in which the infrared cameras 10 or the like capture images of the subject P using the light beams having multiple wavelengths. This control program may be stored in a computer-readable storage medium, such as an optical disk, CD-ROM, USB memory, or SD card and then provided.

### DESCRIPTION OF REFERENCE SIGNS

P . . . subject, SYS1 to SYS10 . . . imaging system, 10, 10a to 10f . . . infrared camera, 100 . . . small infrared camera, 20, 20a to 20f, 200 . . . lighting unit, 30 . . . control unit, 31 . . . image processing unit, 32 . . . lighting drive unit, 41, 41a, 41b, 41c . . . visible camera, 42 . . . visible lighting unit, 50 . . . drive unit

What is claimed is:

1. An imaging system comprising:
an infrared camera that is sensitive to light beams having wavelengths in an infrared region;
a lighting unit that emits light beams having a plurality of wavelengths in an infrared region in a region including the wavelengths to which the infrared camera is sensitive; and a control unit that controls capture of an image by the infrared camera and emission of a light beam by the lighting unit.

2. The imaging system of claim 1, wherein the lighting unit emits light beams having a plurality of wavelengths of 800 nm or more and 2500 nm or less.

3. The imaging system of claim 2, wherein the lighting unit emits light beams having a plurality of wavelengths of 1000 nm or more and 1600 nm or less.

4. The imaging system of claim 1, further comprising:
a visible camera that is sensitive to a light beam having a wavelength in a visible region; and
a visible lighting unit that emits a light beam having the wavelength to which the visible camera is sensitive.

5. The imaging system of claim 1, wherein the control unit comprises a lighting drive unit that causes the lighting unit to emit light beams having different wavelengths sequentially or simultaneously.

6. The imaging system of claim 5, wherein the control unit synchronizes switching between emission wavelengths by the lighting drive unit and capture of an image by the infrared camera.

7. The imaging system of claim 1, wherein the control unit assigns a frame number to each wavelength in a manner corresponding to the degrees of sensitivity of the infrared camera to the wavelengths.

8. The imaging system of claim 1, wherein the infrared camera comprises a plurality of infrared cameras disposed in such a manner that the infrared cameras look into an identical portion of a subject in different fields of view; and
the lighting unit comprises lighting units disposed so as to correspond to the infrared cameras, wherein
the infrared cameras capture images of light beams emitted from the lighting units and then reflected from the subject or light beams emitted from the lighting units and then transmitted through the subject.

9. The imaging system of claim 1, further comprising a drive unit that moves the infrared camera and the lighting unit so that the infrared camera looks into an identical portion of a subject in different fields of view.

10. The imaging system of claim 1, wherein the control unit comprises an image processing unit that combines a plurality of images captured by the infrared camera.

11. The imaging system of claim 10, wherein the image processing unit generates a stereoscopic image of the subject on the basis of the images captured by the infrared camera.

12. The imaging system of claim 1, wherein the lighting unit emits the light beams having the wavelengths comprising at least one infrared wavelength of 1000 nm or more and is mounted on at least one of a mammotome device, mammography, a dermoscope, and a tooth row transmission device.

13. An imaging method comprising:
emitting light beams having a plurality of wavelengths in an infrared region toward a subject; and
capturing images of the subject using the light beams having the wavelengths.

14. An imaging system for capturing an image of a living body tissue, comprising:
a lighting unit that emits infrared light beams having wavelengths in an infrared region based on spectral properties of water and lipid;
an infrared camera that receives the infrared light beams;
a visible lighting unit that emits a visible light beam having a wavelength in a visible region;
a visible camera that receives the visible light beam; and
a control unit comprising an image processing unit that processes an infrared image captured by the infrared camera using a visible image captured by the visible camera.

15. The imaging system of claim 14, wherein the infrared camera is sensitive to the infrared light beams in a wavelength region of 1000 nm or more and 1600 nm or less.

16. The imaging system of claim 14, wherein the lighting unit emits the infrared lights having predetermined wavelengths in a wavelength region of 1000 nm or more and 1600 nm or less.

17. The imaging system of claim 14, wherein the control unit comprises a lighting drive unit that causes the infrared light beams and the visible light beam to be emitted sequentially or simultaneously.
